(19) United States
(12) Patent Application Publication
(10) Pub. No.: US 2015/0282837 A1
(43) Pub. Date: Oct. 8, 2015

(54) MODULE FOR ADJUSTING LAUNCH OF LANCET, LANCET DEPTH ADJUSTMENT DEVICE, AND LANCING DEVICE COMPRISING SAME

(71) Applicant: In Sang CHOI, (US)

(72) Inventor: In Sang Choi, Anyang-si, Gyeonggi-do (KR)

(21) Appl. No.: 14/437,330

(22) PCT Filed: Oct. 22, 2013
(86) PCT No.: PCT/KR2013/009407

§ 371 Date: Apr. 21, 2015

(30) Foreign Application Priority Data
Oct. 23, 2012 (KR) 10-2012-0117725
Oct. 23, 2012 (KR) 10-2012-0117727

(51) Int. Cl. A61B 17/34

(52) U.S. Cl. CPC A61B 17/3494

(57) ABSTRACT

A module for adjusting the launch of a includes: a cam plate slidingly moving longitudinally having a first and second catching parts; a cap lever rotatably coupled to a rotating shaft formed in parallel with the cam plate movement direction, and having a first protrusion moving while being inserted in the first catching part and a cap arm for catching a protective cap; and a collet lever rotatably coupled to the rotating shaft having a second protrusion moving while being inserted in the second catching part and a body arm for catching a lancet body. When the cam plate moves backward to a first point, the collet lever moves backward with respect to the cap lever separating the protective cap from the lancet body. When the cam plate moves backward to a second point, the cap lever and the collet lever rotate so that the lancet body moves forward.

### TECHNICAL FIELD

The present invention relates to a module for adjusting launch of a lancet, a lancet depth adjustment device, and a lancing device including the same. More particularly, the present invention relates to a lancet depth adjustment device usable in a blood glucose measurement device configured to cause a lancet and a strip to protrude to measure blood glucose, a module for adjusting launch of the lancet which is proper to manufacture a small lancing device and is convenient to use and a lancing device including the same.

### BACKGROUND ART

A lancing device is generally configured to take blood and return to the original position thereof using elastic force of a spring in order to test blood glucose in a body.

Specifically, after a cap is removed from the lancing device, a separate lancet is inserted through an insertion hole. Then, a protection cap of the lancet is removed and the cap of the lancing device is reconnected. Thereby, when a button in the lancing device is pushed, the lance is moved down into a body by operation of the spring.

Thereafter, the cap of the lancing device removed, the protection cap of the lancet is reconnected, and then the lancet is removed from the insertion hole.

Accordingly, the lancing device and the lancet always need to be inconveniently carried together. In addition, it is inconvenient to manually insert the lancet into the insertion hole.

To address these problems, Korean Patent No. 10-1177260 discloses “APPARATUS FOR AUTOMATICAL DETECTING BLOOD.” Specifically, a lancet is guided to and inserted into an insertion groove by a lancet guide member. The inserted lancet is moved downward and fixed by a lancet fixing member to take blood. After blood is taken, the lancet is taken away from the insertion groove and discharged outside by a lancet discharge member configured to transport the lancet at a predetermined angle. As a series of operations of inserting the lancet into the groove and discharging the lancet therefrom are automatically performed, blood can be conveniently taken.

For such lancing device, which is intended to be carried by a user and immediately used when necessary, portability is very important. For the automatic lancing device of Korean Patent No. 10-1177260, a lancet guide member, a lancet fixing member, a lancet discharge member are engaged with each other according to rotation of a rotation member. Since the diameter of the rotation member cannot be reduced, the lancing device is large in volume and improper for manufacturing a compact, particularly, slim lancing device.

Meanwhile, a lancing device of the conventional art is significantly inconvenient to use since the user needs to insert the lance into the lancing device after removing the protection cap from the lancet and to reconnect the lancet to the protection cap and remove the same after blood is taken. Therefore, the lancing device needs to be improved.

In the conventional art of lancing devices including Korean Patent No. 10-1177260, a lance for taking blood and a strip for measuring blood glucose protrude individually, or a specific means to adjust the degree of protrusion of the lancet is not disclosed. In other words, the degree of protrusion of the lancet needs to be adjusted in order to properly take blood with minimized pain caused to the user, but the conventional art fails to disclose a means to adjust lancet depth, which needs to be improved.

### DISCLOSURE

### Technical Problem

An object of the present invention is to provide a module for adjusting launch of a lancet capable of automatically performing a series of operations including separating a protection cap from the lancet for use and reconnecting the protection cap to the lancet after use of the lancet and maximizing portability and convenience by allowing for manufacturing of a lancing device that is compact, particularly, slim, and a lancing device including the same.

Another object of the present invention is to provide a lancet depth adjustment device that is applied to a device having a protrusible strip and a protrusible lancet as a device used to measure blood glucose and is capable of adjusting the lancet depth by controlling the length of protrusion of the lancet in connection with protrusion of the strip, and a lancing device including the same.

### Technical Solution

The objects of the present invention can be achieved by providing a lancet depth adjustment device including a strip withdrawer allowing a strip to straightly protrude therethrough, a supply lever configured to slidably move in connection with the strip in a direction parallel to a protrusion direction of the strip, a controller configured to control a degree of movement of the strip and the supply lever, and a launcher provided with a lancet coupled to one side thereof and configured to move in a direction parallel to the protrusion direction of the strip, wherein a range of movement of the launcher is limited by the supply lever.

The movement of the strip and supply lever may be caused by conversion of rotational motion of a drive motor into reciprocation.

The strip may protrude as the supply lever moves forward.

The supply lever may be provided with a stopping arm protruding in a direction perpendicular to a movement direction of the strip, wherein the launcher may be provided with a catch projection, the catch projection being caught by the stopping art when the launcher moves forward.

When the supply lever moves backward, the launcher may move backward with the catch projection caught by the stopping arm.

A front end of the launcher may be provided with a lancet coupler, the lancet being coupled to the lancet coupler.

The objects of the present invention can also be achieved by providing a lancing device including the lancet depth adjustment device.

Herein, the lancing device may further include a module for adjusting launch of a lancet, wherein the module may include a cam plate configured to slidably move forward and backward in a longitudinal direction, the cam plate including a first catch part and a second catch part, a cap lever pivotably coupled to a pivot axis arranged parallel to a movement direction of the cam plate, the cap lever including a first projection movably inserted into the first catch part and a cap arm configured to catch the protection cap, and a collet lever pivotably coupled to the pivot axis, the collet lever including a second projection movably inserted into the second catch part and a body arm configured to catch a lancet body, wherein, when the cam plate moves backward to a first point, the collet lever may move backward with respect to the cap lever and the protection cap may be separated from the lancet body, wherein, when the cam plate moves backward to a second point, the cap lever and collet lever may rotate to allow the lancet body to move forward.

The cam plate may include a third catch part arranged behind the second catch part, and the lancing device may further include a launch lever pivotably coupled to the pivot axis, the launch lever including a third projection movably inserted in the third catch part and a launch arm configured to catch the launcher, wherein, when the cam plate moves to the first point, the lancet body may be coupled to the launcher, wherein, when the cam plate moves to the second point, the lancet body may move forward along with the launcher.

The first catch part may include a first straight part arranged parallel to the movement direction of the cam plate, a 1-1st inclined part inclined forward and upward at a front end of the first straight part and a 1-2nd inclined part inclined backward and upward at a rear end of the first straight part.

The cap lever may include a pivot slot part allowing the second projection to be inserted thereinto, the pivot slot part extending in a longitudinal direction of the cam plate.

The second catch part may include a 2-1st inclined part inclined forward and upward and a 2-2nd inclined part inclined backward and upward at a rear end of the 2-1st inclined part.

The third catch part may include a third inclined part inclined forward and upward and a third straight part extending backward from a rear end of the third inclined part.

The cam plate may be formed in a plate shape, and the first catch part, second catch part and third catch part may be arranged in order from a front to back of the cam plate.

A surface of the cam plate may be provided with a rack gear, the first catch part and second catch part and third catch part being formed on the surface, wherein the cam plate may be moved forward and backward by rotation of a drive gear engaged with the rack gear.

### Advantageous Effects

According to an embodiment of the present invention, as a cam plate slidably moves back and forth, a cap lever, a collet lever and the like are allowed to pivotably move. Accordingly, the thickness of the cam plate can be reduced. Thereby, a small lancing device can be manufactured. In addition, since a protection cap can be separated from and coupled to a lancet body by engagement of the cam plate, cap lever and collet lever, convenience can be maximized in using the lancing device.

According to an embodiment of the present invention, a device used to withdraw a strip for measuring blood glucose is also used to adjust the degree of protrusion of a lancet. Thereby, the lancet depth can be easily adjusted. In addition, a separate means to adjust the lance depth does not need to be provided in a device for measuring blood glucose in forming the device, and therefore the device for measuring blood glucose can have a simple stricter and exhibit excellent performance.

### DESCRIPTION OF DRAWINGS

FIG. 1 is a plan view illustrating a module for adjusting launch of a lancet connected to a baseplate according to an embodiment of the present invention.

FIG. 2 is a perspective view illustrating the module for adjusting launch of a lancet shown in FIG. 1.

FIG. 3 is a side view illustrating the module for adjusting launch of a lancet shown in FIG. 2.

FIG. 4 is a side view illustrating a cam plate shown in FIG. 1.

FIG. 5 is a perspective view illustrating a lancet depth adjustment device according to an embodiment of the present invention.

FIG. 6 is a plan view illustrating the lancet depth adjustment device shown in FIG. 5.

FIG. 7 is a side view illustrating the lancet depth adjustment device shown in FIG. 5.

### DESCRIPTION OF REFERENCE NUMERALS OF MAIN PARTS IN THE DRAWINGS

1: Module for adjusting launch of a lancet
10: Cam plate
11: First catch part
11a: 1-1st inclined part
11b: First straight part
11c: 1-2nd inclined part
12: Second catch part
12a: 2-1st inclined part
12b: 2-2nd inclined part
13: Third catch part
13a: Third inclined part
13b: Third straight part
14: Rack gear
20: Cap lever
21: First projection
22: Cap arm
23: Pivot slot part
30: Collet lever
31: Second projection
32: Body arm
40: Launch lever
41: Third projection
42: Launch arm
50: Launcher
52: Elastic member
53: Lancet coupler
54: Catch projection
60: Lancet assembly
61: Protection cap
62: Lancet body
70: Baseplate
101: Lancet depth adjustment device
110: Strip withdrawer
120: Supply lever
121: Stopping arm
130: controller
140: Strip
170: Gear
180: Drive motor
A: Pivot axis
P0: Zero point
P1: First point
P2: Second point
P3: Third point

### BEST MODE

Reference will now be made in detail to the preferred embodiments of the present invention, examples of which are illustrated in the accompanying drawings. In describing the present invention, descriptions of well-known technologies may be omitted in order not to obscure the gist of the present invention.

As described herein, a module for adjusting launch of a lancet 1, which is used o tech blood glucose, constitutes a lancing device configured to take blood to test blood glucose. A lancet depth adjustment device 101 constitutes the lancing device and a blood glucose measurement device.

Accordingly, in the description given below, the lancing device may have the same meaning as the blood glucose measurement device.

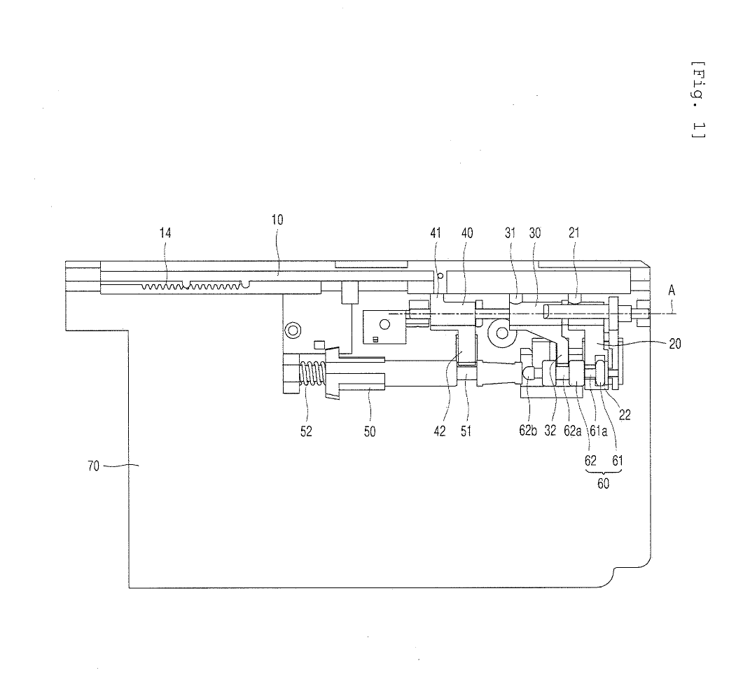

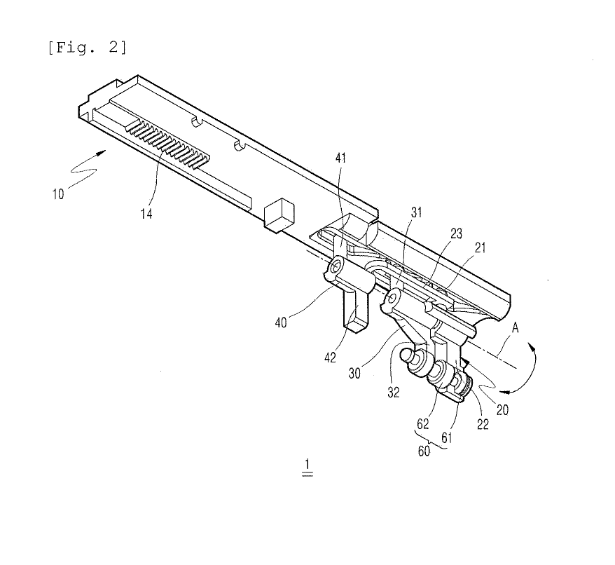

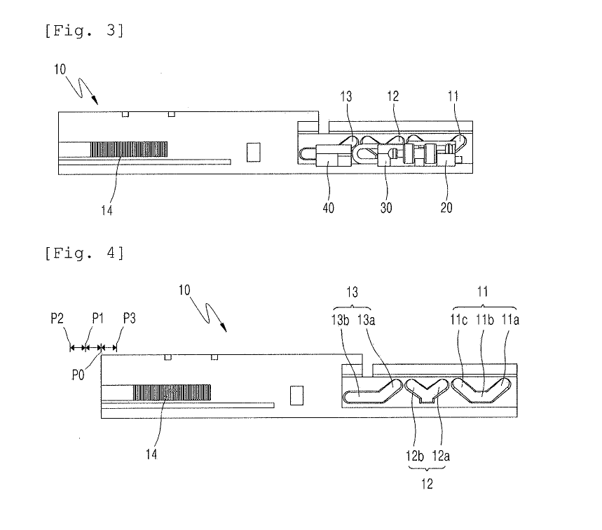

FIG. 1 is a plan view illustrating a module 1 for adjusting launch of a lancet connected to a baseplate 70 according to an embodiment of the present invention, and FIG. 2 is a perspective view illustrating the module 1 for adjusting launch of a lancet shown in FIG. 1. FIG. 3 is a side view illustrating the module 1 for adjusting launch of a lancet shown in FIG. 2, and FIG. 4 is a side view illustrating a cam plate 10 shown in FIG. 1.

In the description given below, directions are defined based on FIG. 1. In FIG. 1, the forward direction points to the right side of the figure, the backward direction points to the left side of the figure. The upward direction points to the viewer of the figure, and the downward direction points down into the figure.

The module 1 for adjusting launch of a lancet constitutes a lancing device configured to take blood to test blood glucose. In particular, the module 1 is a part of a device configured to perform a series of operations including removing a protection cap 61 from a lancet body 62 coupled with a lancet, launching the lancet and reconnecting the protection cap. The parts of the lancing device except the module 1 may be configured to have the same structure and operations as those of a typical lancing device.

The module 1 includes a cam plate 10, a cap lever 20, a collet lever 30 and a launch lever 40.

In this specification, the assembly of the lancet body 62 and the protection cap 61 will be referred to as a lancet assembly 60. A sharp lancet (not shown) is coupled to the front of the lancet body 62, and the protection cap 61, which accommodates the lancet and prevents the lancet from being exposed outside, is coupled to the lancet body 62. The protection cap 61 is formed such that the back 61a of the protection cap 61 has a smaller diameter than the front thereof, and the lancet body 62 is formed such that the middle part 62a of the lancet body 62 has a smaller diameter than the other part thereof.

The cam plate 10 is generally formed in a flat and long plate shape. Formed on one side surface of the cam plate 10 are a first catch part 11, a second catch part 12, a third catch part 13 and a rack gear 14. In forming the lancing device, the cam plate 10 is positioned to one side of the baseplate 70 and perpendicularly connected to the baseplate 70.

With the cam plate 10 connected to the baseplate 70, the first catch part 11, the second catch part 12, the third catch part 13 and the rack gear 14 are arranged on the inner surface of the cam plate 10. As the cam plate 10 slidably moves back and forth according to rotation of a drive gear (not shown) engaged with the rack gear 14, the cap lever 20, the collet lever 30 and the launch lever 40 rotates in operative connection with each other. Thereby, the lancet body 62 and the protection cap 61 are separated and coupled, and a launcher 50 is controlled. Since the cam plate 10 is formed in the shape of a flat plate, and the lancing device operates as the cam plate slidably moves back and forth, the size, thickness and width of the cam plate 10 may be significantly reduced, and the area occupied by the cam plate 10 during movement may also be reduced. Accordingly, a downsized slip lancing device may be manufactured.

The first catch part 11, the second catch part 12 and the third catch part 13 are arranged in order from the front to back of the cam plate 10. The catch parts are formed to guide movements of the cap lever 20, the collet lever 30 and the launch lever 40, respectively. The first catch part 11, the second catch part 12 and the third catch part 13 may have a concave groove shape as shown in FIGS. 2 to 4, and have various shapes within a range allowing for guidance of movement of the cap lever 20, the collet lever 30 and the launch lever 40.

Specifically, the first catch part 11 includes a 1-1st inclined part 11a, a first straight part 11b and a 1-2nd inclined part 11c. The first straight part 11b is arranged in parallel with the movement direction of the cam plate 10, the 1-1st inclined part 11a is inclined upward at the front end of the first straight part 11b, and the 1-2nd inclined part 11c is inclined upward at the rear end of the 1-1st straight part. Preferably, the 1-2nd inclined part 11c and the 1-2nd inclined part 11c are symmetrically formed with respect to the center of the 1-1st straight part. Preferably, the first straight part lib is dented with a depth greater than or equal to that of the 1-1st inclined part 11a and the 1-2nd inclined part 11c in order to ensure smooth guidance of a first projection 21, which will be described later.

Next, the second catch part 12 includes a 2-1st inclined part 12a and a 2-2nd inclined part 12b. The 2-1st inclined part 12a is inclined forward and upward, and the 2-2nd inclined part 12b is inclined upward at the rear end of the 2-1st inclined part 12a. Preferably, the 2-1st inclined part 12a has a length, size, and inclination direction which are the same as or correspond to the length, size and inclination direction of the 1-1st inclined part 11a, and the 2-2nd inclined part has a length, size, and inclination direction which are the same as or correspond to the length, size and inclination direction of the 1-2nd inclined part 11c.

The third catch part 13 includes a third inclined part 13a and a third straight part 13b. The third inclined part 13a is inclined forward and upward, and the third straight part 13b is extended rearward from the rear end of the third inclined part 13a and is arranged in parallel with the movement direction of the cam plate 10. Preferably, the third inclined part 13a has a length, size, and inclination direction which are the same as or correspond to the length, size and inclination direction of the 1-1st inclined part 11a and the 2-1st inclined part 12a. The third straight part 13b is longer than the first straight part 11b. Preferably, the length of the third straight part 13b is about twice the length of the first straight part 11b.

The cap lever 20 is coupled to the cam plate 10 so as to rotate about a pivot axis A arranged parallel to the movement direction of the cam plate 10. The cap lever 20 includes a first projection 21, a cap arm 22 and a pivot slot part 23. The cap lever 20 cannot fully rotate about the pivot axis A, but can rotate downward within a predetermined range in a reciprocating manner.

The first projection 21 is inserted into the first catch part 11 to slidably move therein. The first projection 21 is fittingly inserted into the 1-1st inclined part 11a, the first straight part 11b and the 1-2nd inclined part 11c to move along the 1-1st inclined part 11a, the first straight part 11b and the 1-2nd inclined part 11c. As the first projection 21 inserted into the 1-1st inclined part 11a, the first straight part lib and the 1-2nd inclined part 11c moves, the cap lever 20 rotates about the pivot axis A within a predetermined range of angle.

The cap arm 22 is formed approximately on the opposite side of a part of the cap lever 20 that is connected to the pivot axis A with respect to the side of the part of the cap lever 20 on which the first projection 21 is formed. The cap arm 22 is formed to be coupled to the protection cap 61 by surrounding a portion of the protection cap 61. As the cap arm 22 is provided with a groove to surround a portion of the protection cap 61, the protection cap 61 is caught by the cap arm 22.

The pivot slot part 23 extends toward the collet lever 30 to allow a second projection 31, which will be described later, to be inserted thereinto. Accordingly, the pivot slot part 23 has a long slot extending in the movement direction of the cam plate 10, and the second projection 31 is inserted in this slot.

The collet lever 30 is coupled to rotate about the pivot axis A and is provided with a second projection 31 and a body arm 32. The collet lever 30 cannot fully rotate about the pivot axis A, but can rotate downward within a predetermined range in a reciprocating manner.

The second projection 31 is inserted into the second catch part 12 to slidably move therein. The second projection 31 is fittingly inserted into the 2-1st inclined part 12a and the 2-2nd inclined part 12b to move along the 2-1st inclined part 12a and the 2-2nd inclined part 12b. As the second projection 31 inserted into the 2-1st inclined part 12a and the 2-2nd inclined part 12b moves, the collet lever 30 rotates about the pivot axis A within a predetermined range of angle.

The body arm 32 is formed approximately on the opposite side of a part of the collet lever 30 that is connected to the pivot axis A with respect to the side of the part of the collet lever 30 on which the second projection 31 is formed, such that the lancet body 62 is caught by the body arm 32. As described above, the lancet body 62 has a middle part which has a reduced diameter. As an end of the body arm 32 is inserted into the middle part, and thus the lancet body 62 is held by the body arm 32.

The launch lever 40 is coupled to rotate about the pivot axis A and is provided with a third projection 41 and a launch arm 42. The launch lever 40 cannot fully rotate about the pivot axis A, but can rotate downward within a predetermined range in a reciprocating manner.

The third projection 41 is inserted into the third catch part 13 to slidably move therein. The third projection 41 is fittingly inserted into the third inclined part 13a and the third straight part 13b to move along the third inclined part 13a and the third straight part 13b. As the third projection 41 inserted into the third inclined part 13a and the third straight part 13b moves, the launch lever 40 rotates about the pivot axis A within a predetermined range of angle.

The launch arm 42 is formed approximately on the opposite side of a part of the cap lever 20 that is connected to the pivot axis A with respect to the side of the part of the cap lever 20 on which the third projection 41 is formed, such that the launcher 50 is caught by the launch arm 42.

The launcher 50 is coupled to the lancet body 62 to move forward. To this end, the rear end of the launcher 50 is provided with an elastic member 52 such as a coil spring which produces elastic force when compressed. When the means to stop forward movement of the launcher 50 is excluded, the launcher 50 is moved forward by the elastic force.

The front end of the launcher 50 is formed to allow the rear end 62b of the lancet body 62 to be coupled thereto in an inserting manner. Specifically, the front end of the launcher 50 is elastically deformable (and may be formed of a material such as plastics or metal). In particular, a part of the front end of the launcher 50 is cut away to allow the rear end of the lancet body 62 to be fixed (by friction) by being naturally inserted into the front end and to be decoupled from the launcher 50.

One side 51 of the launcher 50 is formed such that the diameter thereof decreases. Thereby, the launch arm 42 is inserted into the launcher 50 and caught by the one side 51.

Hereinafter, description will be given of operation of the module 1 for adjusting launch of a lancet.

Before the lancing device operates, the first projection 21 is positioned at the rear end of the first straight part 11b, the second projection 31 is positioned at the center of the first catch part 11, i.e., between the 2-1st inclined part 12a and the 2-2nd inclined part 12b, and the third projection 41 is positioned at the center of the third straight part 13b.

When an operation signal is input by a user, a drive motor provided in the lancing device rotates, and a drive gear (not shown) receives rotational power transferred from the drive motor and rotates. Then, the cam plate 10 is moved backward by the rack gear 14 engaged with the drive gear. That is, the direction of rotation of the drive motor and drive gear are controlled such that the cam plate 10 can move backward.

Then, the cam plate 10 is slidably moved backward from the zero point P0 to a first point P1. Herein, the first point P1, which is a point located behind the zero point P0, has a relative distance value indicating a distance by which the cam plate 10 is moved. Preferably, the first point P1 has a value corresponding to the length of the first straight part 11b.

As the cam plate 10 moves to the first point P1, the first projection 21 moves all the way to the front end of the first straight part 11b, and the third projection 41 moves all the way to the front end of the third straight part 13b. Since the second projection 31 is inserted in the slot formed in the pivot slot part 23 of the cap lever 20, the second projection 31 cannot move along the 2-1st inclined part 12a, but moves backward while being positioned between the 2-1st inclined part 12a and the 2-2nd inclined part 12b.

Thereby, the cam plate 10 and the collet lever 30 move backward by a distance corresponding to the first straight part 11b, while the cap lever and the launch lever 40 remains stationary. Additionally, as the lancet body 62 moves backward with the protection cap 61 remaining stationary, the protection cap 61 is separated from the lancet body 62. And the rear end of the lancet body 62 is coupled to the front end of the launcher 50.

Next, as the drive motor further rotates, the drive gear receiving rotational power from the drive motor rotates, and the cam plate 10 is moved further backward by the rack gear 14 engaged with the drive gear. Thereby, the cam plate 10 slidably moves backward all the way to a second point P2. Herein, the second point P2, which is a point located behind the first point P1, has a relative distance value indicating a distance by which the cam plate 10 moves. Preferably, the second point P2 has a value corresponding to the horizontal length of the 1-1st inclined part 11a, the 2-1st inclined part 12a or the third inclined part 13a.

As the cam plate 10 moves to the second point P2, the first projection 21 moves all the way to the front end of the 1-1st inclined part 11a, and the third projection 41 moves all the way to the front end of the third inclined part 13a. Since the second projection 31 is inserted in the slot formed in the pivot slot part 23 of the cap lever 20 and the pivot slot part rotates along with the cap lever 20, the second projection 31 moves along the 2-1st inclined part 12a.

Thereby, all of the cap lever 20, the collet lever 30 and the launch lever 40 rotate downward. Specifically, as the cap lever 20 retaining the protection cap 61 rotates downward and the collet lever 30 rotates downward, the lancet body 62 is released. As the launch lever 40 rotates downward, the launcher 50 is released.

As a result, the lancet body 62 moves forward along with the launcher 50, and the lancet is exposed outside the lancing device to take blood.

After blood is taken, the lancet body 62 coupled to the lancet is retrieved into the lancing device and is then coupled to the protection cap 61, which will be described below.

When the launcher 50 coupled to the lancet body 62 is pressed by a separate means (not shown), the launcher 50 is introduced into the lancing device and moved backward to a position where the lancet body 62 is launched.

Next, the drive motor rotates in a direction opposite to the direction of rotation described above, and the cam plate 10 is moved forward by the rack gear 14 engaged with the drive gear. Thereby, the cam plate 10 slidably moves forward all the way to the first point P1. Then, the protection cap 61 is caught by the cap arm 22, the lancet body 62 is caught by the body arm 32, and the launcher 50 is caught by the launch arm 42. Herein, the lancet body 62 is separated from the protection cap 61.

As the drive motor further rotates, the cam plate 10 is moved further forward to the original position P0 by the rack gear 14 engaged with the drive gear. At this time, the protection cap 61 is caught by the cap arm 22, the lancet body 62 is caught by the body arm 32, and the launcher 50 is caught by the launch arm 42. Thereby, the lancet body 62 is coupled to the protection cap 61.

According to the embodiment described above, as the cam plate 10 is configured to slidably move back and forth to rotate the cap lever 20 and the collet lever 30, the cam plate 10 can be formed to be thin. Thereby, a compact lancing device can be manufactured. In addition, since the protection cap 61 can be separated from and coupled to the lancet body 62 by interoperation of the cam plate 10, the cap lever 20 and the collet lever 30, convenient can be maximized in suing the lancing device.

For the lancing device of the present invention, the lancet can be reused after it is used (i.e., after the lancet is exposed outside the lancing device), or can be directly discharged from the lancing device. When the lancet is reused, the operations described above are repeated without discharging the lancet body 62 and the protection cap 61. Hereinafter, a brief description will be give of the process of discharging the lancet.

When a discharge signal is input by the user, the drive motor provided in the lancing device rotates, the cam plate 10 is moved forward by the rack gear 14 engaged with the drive gear. That is, the direction of rotation of the drive motor and drive gear are controlled such that the cam plate 10 can move forward.

Thereby, the cam plate 10 moves forward all the way to a third point P3. As the cam plate 10 moves forward, the first projection 21 moves all the way to the rear end of the 1-2nd inclined part 11c, and the third projection 41 moves all the way to the rear end of the third straight part 13b. Since the second projection 31 remains inserted into the slot formed in the pivot slot part 23 of the cap lever 20, the second projection 31 moves all the way to the rear end of the 2-2nd inclined part 12b while being restricted by the pivot slot part 23.

Thereby, the cap lever 20 and the collet lever 30 rotates downward, the protection cap 61 and the lancet body 62 are released, and the launcher 50 remains caught by the launch lever 40. As a result, the lancet assembly 60 formed by coupling the lancet body 62 and the protection cap 61 are discharged from the lancing device by a separate discharge means without being restricted by any other elements in the lancing device.

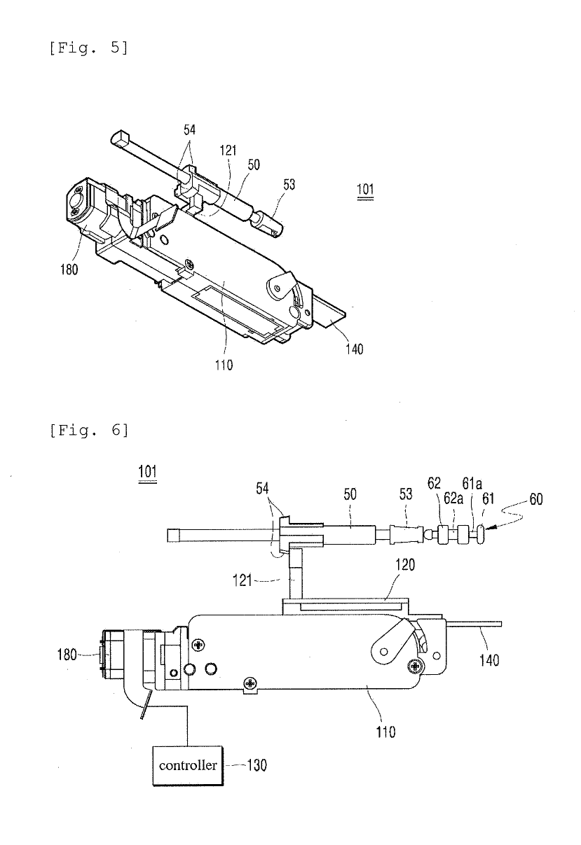

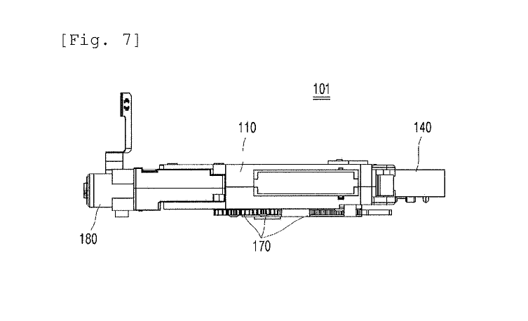

FIG. 5 is a perspective view illustrating a lancet depth adjustment device 101 according to an embodiment of the present invention, FIG. 6 is a plan view illustrating the lancet depth adjustment device 101 shown in FIG. 5, and FIG. 7 is a side view illustrating the lancet depth adjustment device 101 shown in FIG. 5.

In describing the lancet depth adjustment device 101, directions are defined based on FIG. 6. The forward direction points to the right side, and the backward direction points to the left side. The upward direction points to the viewer of the figure, and the downward direction points down into the figure.

The lancet depth adjustment device 101 constitutes a blood glucose measurement device configured to take blood to test blood glucose. In particular, the lancet depth adjustment device 101 constitutes a device for withdrawing a strip 140 used to measure blood glucose. The parts of the blood glucose measurement device except the lancet depth adjustment device 101 may be configured to have the same structures and operations as a common measurement device.

In this embodiment, a lancet is directly or indirectly coupled to the launcher 50 and moved forward and backward along with the launcher 50.

The lancet may be coupled directly to the launcher 50. That is, the lancet have a sharp shape may be coupled to the launcher 50 such that the lancet is exposed outside the launcher 50.

Alternatively, the lancet may be coupled to the lancet assembly 60 as shown in FIG. 6. The lancet assembly 60 is formed by coupling the lancet body 62 and the protection cap 61.

As described above, the back 61a of the protection cap 61 has a smaller diameter than the front thereof, the middle part 62a of the lancet body 62 has a reduced diameter. This is intended to cause the lancet to be exposed as the protection cap 61 is separated from the lancet body 62. The protection cap 61 and the lancet body 62 are separated from each other while being retained by separately provided means.

The lancet depth adjustment device 101 includes a strip withdrawer 110, a supply lever 120, a controller 130 and a launcher 50. A strip 140 is a member by which blood is absorbed or adsorbed. The strip 140 is a means capable of measuring blood glucose using photometry or electrochemical measurement. The strip 140, which is applied to a blood glucose measurement device for measuring a blood glucose level of a diabetic patient, is installed in the blood glucose measurement device and is configured to automatically protrude outward.

The strip 140 is fixed to one side of the strip withdrawer 110 or installed therein, and is withdrawn forward by operation of a drive motor 180. In other words, in the case where the lancet depth adjustment device 101 constitutes the blood glucose measurement device, the strip 140 is accommodated in the blood glucose measurement device until measurement of blood glucose is performed, and is withdrawn out of the blood glucose measurement device when the measurement is performed.

The supply lever 120, is coupled to one side of the strip withdrawer 110, slidably moves back and forth with respect to the strip withdrawer 110. Movement of the supply lever 120 is caused by rotation of the drive motor 180. That is, the rotational motion of the drive motor 180 is converted into rectilinear motion and transferred to the supply lever 120. To this end, a combination of multiple gears 170 may be provided. The gears 170 may be connected to the supply lever 120 by a common structure such as a rack and pinion.

The supply lever 120 is provided with a stopping arm 121, which protrudes in a direction perpendicular to the direction of movement of the strip 140. The stopping arm 121 serves as a stopper when the launcher 50 moves. The stopping arm 121 stops forward movement of the launcher 50 beyond a predetermined range by blocking one side of the launcher 50.

The movement of the strip 140 and the movement of the supply lever 120 are simultaneously caused. In this case, a means for forward/backward movement of the strip 140 may be identical to a means for movement of the supply lever 120, or may be separate from a means for movement of the supply lever 120.

In the former case, the strip 140 may be fixed or coupled to one side of the supply lever 120 to slidably move back and forth along with the supply lever 120. Alternatively, the strip 140 may not coupled to the supply lever 120, but the power according to rotational motion of the drive motor 180 may be converted into rectilinear motion and transferred to each of the strip 140 and the supply lever 120.

In the latter case, the means for forward/backward movement of the strip 140 is distinguished from the means for forward/backward movement of the supply lever 120. For example, a means for transferring power to the supply lever 120 is provided outside the withdrawer, and a means for transferring power to the strip 140 may be provided inside the withdrawer.

In any cases, the strip 140 and the supply lever 120 simultaneously move forward or backward by the same distance.

The controller 130 drives the drive motor 180 and adjusts the degree of rotation of the drive motor 180 according to input information. In other words, when the user inputs information about whether to withdraw the strip 140 or the lancet and the length to be withdrawn, the controller 130 controls rotation of the drive motor 180 according to the gear ratio, and cause the supply lever 120 and the strip 140 to protrude according to the controlled degree of rotation.

The launcher 50 moves in a direction parallel to the direction of protrusion of the strip 140, the range of movement thereof is limited by the supply lever 120. One side of the launcher 50 is provided with a catch projection 54. As the catch projection 54 is stopped by the stopping arm 121 of the supply lever 120, forward movement of the launcher 50 is restricted.

The catch projection 54 also causes the launcher 50 to move backward. When the supply lever 120 having moved forward is moved backward by the drive motor 180, the supply lever 120 moves backward, causing the catch projection 54 to be caught by the drive motor 180. Thereby, the launcher 50 is moved backward. That is, after the lance protrudes and takes blood, the lancet needs to move backward again along with the launcher 50. This movement is caused by the supply lever 120.

Meanwhile, the launcher 50 is configured to be coupled to the lancet body 62 and move forward. To this end, the rear end of the launcher 50 may be provided with an elastic member 52 such as a coil spring capable of producing elastic force by being pressed. When the means to stop forward movement of the launcher 50 is excluded, the launcher 50 is moved forward by the elastic force. If the catch projection 54 is caught by the stopping arm 121 during forward movement of the launcher 50, movement of the launcher 50 is stopped.

The front end of launcher 50 is formed to allow the rear end 62b of the lancet body 62 to be coupled thereto in an inserting manner and is provided with a lancet coupler 53. Specifically, the lancet coupler 53 is elastically deformable (and may be formed of a material such as plastics or metal). In particular, a part of the lancet coupler 53 is cut away to allow the rear end of the lancet body 62 to be fixed (by friction) by being naturally inserted into the front end and to be decoupled from the lancet body 62.

FIG. 6 illustrates the lancet body 62 and the lancet coupler 53, which are not coupled to each other yet. the lancet body 62 and the lancet coupler 53 are coupled as a part of the rear end of the lancet body 62 which has a reduced diameter is inserted into the lancet coupler 53.

Hereinafter, use of the lancet depth adjustment device 101 will be briefly described.

First, the supply lever 120 moves backward, positioning the launcher 50 and the lancet coupled to the front end of the launcher 50 on the rear side.

Next, the user selects an exposure length of the lancet and input corresponding information through the controller 130. Thereby, the strip 140 and the supply lever 120 are slidably moved forward to a predetermined position by operation of the drive motor 180. Herein, forward movement of the launcher 50 is limited by a separately provided means. The launcher 50 moves forward when information corresponding to forward movement is input.

As the launcher 50 is moved forward, the front end of the lancet is exposed outside the blood glucose measurement device. When the catch projection 54 is stopped by the stopping arm 121, forward movement of the launcher 50 and the lancet is stopped. If the protrusion length of the lancet (the length of the lancet protruding outside the blood glucose measurement device) is not proper to take blood (i.e., if the lancet protrudes excessively or insufficiently in view of each user), the user can finely move the supply lever 120 forward or backward through the controller 130 (within a few millimeters) to set a proper protrusion length.

In this case, the protrusion length of the strip 140 also changes within a predetermined range. The strip 140 may protrude a little further or less than the lancet, but this does not affect measurement of blood glucose by the strip 140.

As elements such as the drive motor 180, the withdrawer and the supply lever 120 which are used to withdraw the strip 140 for measurement of blood glucose are used to adjust the degree of protrusion of the lancet, the lancet depth can be readily adjusted. In addition, since a separate means to adjust the lancet depth does not need to be provided in a blood glucose measurement device, the blood glucose measurement device can have a simple structure and exhibit excellent performance.

It will be apparent to those skilled in the art that various modifications and variations can be made in the present invention without departing from the spirit or scope of the invention. Thus, it is intended that the present invention covers the modifications and variations of this invention provided they come within the scope of the appended claims and their equivalents.

### INDUSTRIAL APPLICABILITY

According to an embodiment of a module for adjusting launch of a lancet of the present invention, a lancet depth adjustment device and a lancing device including the same, a device used to withdraw a strip is also used to adjust the degree of protrusion of the lancet. Thereby, the lancet depth can be readily adjusted. In addition, in forming a blood glucose measurement device, a separate means to adjust the lancet depth does not need to be provided in the blood glucose measurement device. Accordingly, the blood glucose measurement device can have a simple structure and exhibit excellent performance. As the present invention overcomes the limit of the prior art, the invention is applicable in the related art and a device to which the present invention is applied is very likely to come to the market. Further, since the present invention can be explicitly practiced in reality, it is industrially available.

1. A lancet depth adjustment device comprising:
a strip withdrawer allowing a strip to straightly protrude therethrough;
a supply lever configured to slidably move in connection with the strip in a direction parallel to a protrusion direction of the strip;
a controller configured to control a degree of movement of the strip and the supply lever; and
a launcher provided with a lancet coupled to one side thereof and configured to move in a direction parallel to the protrusion direction of the strip, wherein a range of movement of the launcher is limited by the supply lever.

2. The lancet depth adjustment device according to claim 1, wherein the movement of the strip and supply lever is caused by conversion of rotational motion of a drive motor into reciprocation.

3. The lancet depth adjustment device according to claim 2, wherein the strip protrudes as the supply lever moves forward.

4. The lancet depth adjustment device according to claim 1, wherein the supply lever is provided with a stopping arm protruding in a direction perpendicular to a movement direction of the strip,
wherein the launcher is provided with a catch projection, the catch projection being caught by the stopping arm when the launcher moves forward.

5. The lancet depth adjustment device according to claim 4, wherein, when the supply lever moves backward, the launcher moves backward with the catch projection caught by the stopping arm.

6. The lancet depth adjustment device according to claim 1, wherein a front end of the launcher is provided with a lancet coupler, the lancet being coupled to the lancet coupler.

7. A lancing device comprising the lancet depth adjustment device according to claim 1.

8. The lancing device according to claim 7, further comprising a module for adjusting launch of a lancet,
wherein the module comprises:
a cam plate configured to slidably move forward and backward in a longitudinal direction, the cam plate comprising a first catch part and a second catch part;
a cap lever pivotably coupled to a pivot axis arranged parallel to a movement direction of the cam plate, the cap lever comprising a first projection movably inserted into the first catch part and a cap arm configured to catch the a protection cap; and
a collet lever pivotably coupled to the pivot axis, the collet lever comprising a second projection movably inserted into the second catch part and a body arm configured to catch a lancet body,
wherein, when the cam plate moves backward to a first point, the collet lever moves backward with respect to the cap lever and the protection cap is separated from the lancet body,
wherein, when the cam plate moves backward to a second point, the cap lever and the collet lever rotate to allow the lancet body to move forward.

9. The lancing device according to claim 8, wherein the cam plate comprises a third catch part arranged behind the second catch part,
the lancing device further comprising a launch lever pivotably coupled to the pivot axis, the launch lever comprising a third projection movably inserted in the third catch part and a launch arm configured to catch the launcher,
wherein, when the cam plate moves to the first point, the lancet body is coupled to the launcher,
wherein, when the cam plate moves to the second point, the lancet body moves forward along with the launcher.

10. The lancing device according to claim 8, wherein the first catch part comprises a first straight part arranged parallel to the movement direction of the cam plate, a 1-1st inclined part inclined forward and upward at a front end of the first straight part and a 1-2nd inclined part inclined backward and upward at a rear end of the first straight part.

11. The lancing device according to claim 10, wherein the cap lever comprises a pivot slot part allowing the second projection to be inserted thereinto, the pivot slot part extending in a longitudinal direction of the cam plate.

12. The lancing device according to claim 8, wherein the second catch part comprises a 2-1st inclined part inclined forward and upward and a 2-2nd inclined part inclined backward and upward at a rear end of the 2-1st inclined part.

13. The lancing device according to claim 9, wherein the third catch part comprises a third inclined part inclined forward and upward and a third straight part extending backward from a rear end of the third inclined part.

14. The lancing device according to claim 9, wherein the cam plate is formed in a plate shape, and the first catch part, second catch part and third catch part are arranged in order from a front to back of the cam plate.

15. The lancing device according to claim 14, wherein a surface of the cam plate is provided with a rack gear, the first catch part and second catch part and third catch part being formed on the surface,
wherein the cam plate is moved forward and backward by rotation of a drive gear engaged with the rack gear.
